(19) United States
(12) Patent Application Publication
(10) Pub. No.: US 2008/0057143 A1
(43) Pub. Date: Mar. 6, 2008

(54) METHOD OF USING COMPOSITION COMPRISING POMEGRANATE EXTRACTS AGAINST INFLUENZA

(76) Inventor: Mohammad MADJID, Houston, TX (US)

Correspondence Address: DALINA LAW GROUP, P.C., 7910 IVANHOE AVE. #325, LA JOLLA, CA, 92037, US

(21) Appl. No.: 11/757,320

(22) Filed: Jun. 1, 2007

Related U.S. Application Data
(60) Provisional application No. 60/809,859, filed on Jun. 1, 2006.

(51) Int. Cl. A61K 36/00

(52) U.S. Cl. 424/769

(57) ABSTRACT

The composition of one or more embodiments of the invention may be used to treat influenza viral infection, since the composition of the present invention has significant antiviral properties as demonstrated by the examples of this application. The composition of the present invention may also be used as a therapeutic composition to treat one or more symptoms of a viral infection, including, but not limited to, sore throat, congestion, laryngitis, mucositis, and/or mucous membrane inflammation by administration to a subject suffering from one or more of these symptoms or ailments.

This application claims benefit of U.S. Provisional Patent Application Ser. No. 60/809,859 filed Jun. 1, 2006, which is incorporated by reference herein.

### BACKGROUND OF THE INVENTION

1. Field of the Invention

One or more embodiments of the invention relate generally to compositions comprising pomegranate extracts and methods for using thereof against influenza viruses.

2. Description of the Related Art

The importance of influenza viruses as worldwide pathogens for humans and domestic animals is well recognized. Influenza is a major cause of morbidity and death. According to the Office of Technology Assessment of the US Congress, each year, in the United States alone, “the flu” accounts for 110,000 hospitalizations, 1 to 3 billion dollars in direct costs, and 10 to 15 billion dollars in indirect costs. Influenza has been established as a serious human affliction that can cause localized epidemics and global pandemic of acute respiratory infection. There is a growing concern for potential pandemic outbreak of influenza virus from the strain currently in birds in Asia or another influenza virus.

Avian influenza is caused by type A strains of influenza virus. Avian influenza occurs throughout the world. Infected birds may display a wide range of symptoms, from a mild illness to a highly contagious fatal disease. The highly contagious disease is caused by an especially virulent strain of influenza virus. Infection by this strain is associated with a sudden onset of severe symptoms, such as a lack of energy, decreased egg production, soft shelled eggs, a swelling of the head, eyelids, etc., nasal discharge, coughing or diarrhea, resulting in death. At present, 15 subtypes have been identified that can infect birds but only H7, H5 and H9 subtypes are associated with outbreaks. The current Asian and British Columbia outbreaks are caused by a H5N1 and H7N3 strains, respectively. As discussed above, influenza viruses are a public health concern because these viruses lack a mechanism for proofreading nucleic acid replication as well as a repair system for correcting such errors. Thus, influenza viruses are especially prone to a high mutation rate during transcription. Additionally, influenza viruses are able to exchange or swap genetic material from other subtypes from different species, thus allowing subtypes to cross the species barrier that normally prevents the cross infection of species specific viruses from one species to another unrelated species. This species barrier normally prevents avian influenza virus strains from infecting humans, but occasionally new strains may have genetic material from both avian and human influenza virus strains. This exchange of genetic material occurs when there is a close proximity between humans and domestic poultry and swine. Swine may act as a reservoir for both human and avian strains. Thus swine act as a natural incubator for the emergence of new strains that can infect humans as well as avian species.

As mentioned above, influenza is prone to minor changes though genetic material to one or more of the major surface antigens during replication. The so-called antigenic drift is responsible for the seasonal epidemics because it can enable the virus to infect persons with only partial immunity from a prior exposure to the virus. Influenza A viruses are especially prone to antigenic drift. Major changes in the H and N antigens result in antigenic shift. Antigenic shift results in a new viral subtype and it can cause major epidemics and pandemics due to minimal immunity in population. Pandemics happen when a novel influenza virus emerges that infects and can be efficiently transmitted between humans. Hence, there is a need for a composition that provides for one or more anti-viral mechanisms that are not affected by antigenic drift.

Influenza viral infection may be associated with redox changes characteristic of oxidative stress. A more oxidized environment may favor further viral infection and stimulates viral protein synthesis. Various studies suggest that apoptosis of mammalian cells may be caused by alteration of intracellular redox condition induced by influenza virus infection. It was recently reported that certain antioxidants inhibited the growth of influenza viruses in Madin-Darby canine kidney (MDCK) cells and in another study, antioxidants inhibited the replication of viral strains in peripheral blood lymphocytes. These substances have proven useful in the field treating various illnesses; however there has not been any progress in the creation of a prophylactic method for use with antioxidant compounds. This lack of effect by other antioxidants on viral titer might represent the difference in biological availability of these compounds or that anti-viral activity operates through mechanisms other than anti-oxidation. Therefore, there exists a need in the field to provide a prophylactic method for the reduction of the incidence of contracting an illness caused by influenza virus.

### BRIEF SUMMARY OF THE INVENTION

Accordingly, it is an object of certain embodiments of the invention to provide a method for reducing the incidence of contracting an illness caused by influenza viruses.

In the first aspect, the present invention relates to a method for the prophylactic use of an anti-viral composition to reduce the incidence of contracting an illness. The method comprises the steps of administering to a subject that has been, or will be, exposed to an illness caused by an influenza virus, an amount of an anti-viral composition having a composition extracted from fruits of pomegranate; and optionally an acceptable carrier. The amount of anti-viral composition is effective, when administered, to reduce the incidence of contracting the illness.

In a second aspect of the invention, a prophylactic anti-viral composition having ingredients obtainable from pomegranate fruit is disclosed. The anti-viral composition is effective, when administered as a nasal spray or as an inhalant spray to a subject that has been, or will be, exposed to an illness caused by influenza virus, to reduce the incidence of contracting said illness.

These and various other advantages and features of novelty that characterize the invention are pointed out with particularity in the claims annexed hereto and forming a part hereof. However, for a better understanding of the invention, its advantages, and the objects obtained by its use, reference should be made to the accompanying descriptive matter, in which there is described a preferred embodiment of the invention.

### BRIEF DESCRIPTION OF THE DRAWINGS

The above and other aspects, features and advantages of the invention will be more apparent from the following more particular description thereof, presented in conjunction with the following drawings wherein:

FIG. 1 is illustrates inhibition of viral replication by PJ after and during viral absorption into cells.

### DETAILED DESCRIPTION

The composition of the present invention may be used to treat influenza viral infection, since the composition of the present invention has significant antiviral properties as demonstrated by the examples of this application. The composition of the present invention may also be used as a therapeutic composition to treat one or more symptoms of a viral infection, including, but not limited to, sore throat, congestion, laryngitis, mucositis, and/or mucous membrane inflammation by administration to a subject suffering from one or more of these symptoms or ailments.

The composition of the present invention may also be employed to reduce the incidence of contracting an illness. In this application of the composition of the present invention, a safe and effective amount of the composition of the present invention is administered to a mammal or a bird that has been or will be exposed to an illness caused by a microbe, to reduce the incidence of contracting said illness, relative to a mammal or a bird that has been or will be exposed to an illness caused by a microbe to which the composition of the present invention has not been administered.

The composition of the present invention may also be formulated with an acceptable carrier. The acceptable carrier may include, but is not limited to: (a) carbohydrates including sweeteners, fructose, sucrose, sugar, dextrose, starch, lactose, maltose, maltodextrins, corn syrup solids, honey solids, commercial tablet nutritional supplements (b) sugar alcohols including mannitol, sorbitol and xylitol; and (c) various relatively insoluble excipients including dicalcium phosphate, calcium sulfate, calcium carbonate, microcrystalline cellulose and other tableting ingredients.

The composition of the present invention may also be formulated into a nasal aerosol or inhalant composition. Such a composition may be prepared using well-known techniques. For these types of formulations, suitable carriers may include the following ingredients: saline with one or more preservatives, absorption promoters to enhance bioavailability, fluorocarbons, and/or conventional solubilizing or dispersion agents.

For the purpose of the invention, an extract from pomegranate may be an extract from the whole pomegranate fruit or from any constituents of pomegranate fruit. Examples of constituents of pomegranate fruit that may be used to make the extract of the invention include, but are not limited to, juice, seed, and the inner and outer peel of pomegranate fruit. In one embodiment of the invention, the extract is the juice extract of whole pomegranate fruit. In another embodiment of the invention, the extract is from the inner or outer peel of pomegranate fruit. In a further embodiment of the invention, the extract may be a mixture of two or more extracts of the whole pomegranate or any constituents of pomegranate.

In general, any methods that may produce pomegranate juice that naturally occurs in pomegranate may be used. For the purpose of the invention, the juice may be concentrated or diluted from its natural concentration. The juice may also be mixed with extracts of other constituents of pomegranate to vary the composition. Methods of making the extract, including the juice from whole pomegranate fruits are described in U.S. Pat. No. 6,977,089 entitled “METHODS OF USING POMEGRANATE EXTRACTS FOR CAUSING REGRESSION IN LESIONS DUE TO ARTERIOSCLEROSIS” which is incorporated herein by reference and in U.S. patent application Ser. No. 11/137,248 entitled “PROCESSES FOR EXTRACTING PHYTOCHEMICALS FROM POMEGRANATE SOLIDS AND COMPOSITIONS AND METHODS OF USE THEREOF” which is also incorporated herein by reference.

Extracts from the constituents of pomegranate, i.e., seeds or the inner or outer peel, may be made by various methods. For example, the seeds or the inner or outer peel of pomegranate may be diluted in water and the extract may be made by crushing, squeezing, or extensive vortexing. The insoluble materials of the extract may be separated from the soluble supernatant of the extract. In some instances, the supernatant of the extract is used for the purpose of the invention, although any oily, lipidic fraction of the extract may also be used. The extract from constituents of pomegranate may be concentrated or diluted, or mixed with each other or with pomegranate juice extract.

In accordance with one embodiment of the invention, the extract of the present invention may be prepared by a process including the steps of: (a) crushing and squeezing the whole fruits of the pomegranate, including the inner and outer peels and the seeds, to yield a juice component and an insoluble by-product component, and (b) separating the juice component from the insoluble by-product component. The juice component may be used as a juice extract of the invention. The insoluble by-product component may be resuspended in an aqueous medium, such as, but not limited to, water or alcohol, and be further crushed, squeezed, and mixed to yield a soluble portion and an insoluble portion. Then the soluble portion may be separated from the insoluble portion to produce the extract of the constituents of the invention. Alternatively, the soluble portion may be combined with the juice extract to produce the extract of the invention.

In one embodiment of the invention, the whole fruit of the pomegranate may be enzymatically treated to improve extraction and filtration. For example, pectinase may be used to treat the whole fruit to prevent the formation of pectin gels. Other enzymes may also be used as long as they can improve extraction and filtration of the extract of the invention.

The extract of pomegranate used in accordance with one or more embodiments of the invention may be in a liquid or solid form. In accordance with one embodiment of the invention, a solid form of the extract may be made by lyophilizing the liquid extract of the invention. Alternatively, the constituents of the pomegranate, such as seeds, inner or outer peels, or any insoluble portion discussed above, may be processed directly to form the solid form of the extract of the invention. For example, the constituents of the pomegranate may be dried, and processed into powder or pill forms to be used directly as the solid form of the extract of the invention.

Compositions of one or more embodiments of the invention may be a variety of kinds, including, but not limited to, nutritional supplements, pharmaceutical preparations, vitamin supplements, food additives, or foods supplements. Compositions of the invention may be in convenient dosage forms, including, but not limited to, tablets, suspensions, implants, solutions, emulsions, capsules, powders, syrups, liquid compositions, ointments, lotions, creams, pastes, gels, or the like.

Compositions of one or more embodiments of the invention may include a carrier. Depending on the kind of compositions of the invention, a carrier may be a dietary suitable carrier or a pharmaceutically acceptable carrier, as long as it is compatible with the particular kind of compositions of the invention. Examples of a dietary suitable carrier include, but are not limited to, dietary suitable excipients, diluents, and carriers. Examples of a pharmaceutically acceptable carrier include, but are not limited to, biocompatible vehicles, adjuvants, additives, and diluents to achieve a composition usable as a dosage form. As used herein, the terms “pharmaceutically acceptable,” “physiologically tolerable,” and grammatical variations thereof, as they refer to compositions, carriers, diluents, and reagents, are used interchangeably and represent that the materials are capable of administration to or upon a mammal without the production of undesirable physiological effects.

The compositions of one or more embodiments of the invention may be used alone or in combination with other biologically active ingredients. A composition of embodiments of the invention, alone or in combination with other active ingredients may be administered to a subject in a single dose or multiple doses over a period of time, generally by oral administration. Various administration patterns will be apparent to those skilled in the art. The dosage ranges for the administration of the compositions of the invention are those large enough to produce the desired effect. The dosage should not be so large as to cause any adverse side effects, such as unwanted cross-reactions and the like. Generally, the dosage will vary with the age, weight, sex, condition, and extent of a condition in a subject, and the intended purpose. The dosage can be determined by one of skill in the art without undue experimentation. The dosage can be adjusted in the event of any counter indications, tolerance, or similar conditions. Those of skill in the art can readily evaluate such factors and, based on this information, determine the particular effective concentration of a composition of the invention to be used for an intended purpose.

In one embodiment of the invention, a composition contains the extract of pomegranate in a dosage unit in an amount that contains at least 30 to 10,000 parts per million or 30 to 3,000 mg of polyphenols. For the purpose of the invention, polyphenols are those naturally present in the extract of pomegranate. It should be appreciated that polyphenols are used herein as a measurement marker for the amount of extract used in each dosage unit. Polyphenols are not used herein as being indicative of the only active, ingredients of the extract. It is possible, for example, that other elements of the composition or the synergy of polyphenols and other components of an extract of the invention, are responsible for the activities of the extract.

The term “dosage unit” as used herein refers to physically discrete units suitable as unitary dosages for subjects, each unit containing a predetermined quantity of active material calculated to produce the desired therapeutic effect in association with the required diluent, e.g., a carrier or vehicle. The specifications for the unit dose of this invention are dictated by and are directly dependent on (a) the unique characteristics of the active material and (b) the limitations inherent in the art of compounding such active material for therapeutical use in subjects.

Prophylactic treatment is aimed at a subject that will soon be exposed to a virus or has recently been exposed to a virus. Such prophylactic treatment may be effective either alone, or to augment a vaccine or another anti-viral drug. Prophylactic treatment may also be used against viruses for which there are not yet a vaccine available. In the case of prophylactic treatment, the composition of the invention is administered to a subject that will be exposed to a virus or has recently been exposed to a virus for the purpose of reducing the incidence of active infection by the virus in that subject.

In another aspect, the present invention relates to a method of reducing, treating or preventing of at least one symptom or adverse effect of viral infection by administering, to a subject infected with a virus, a composition of the present invention, including ingredients that can be obtained from pomegranate.

In the method, the subject may be a human, an in vitro cell system, or an animal. Preferably, the subject is a mammal, more preferably, a human. In the method, the virus that may be inhibited by administration of the composition of the present invention includes, among other viruses, rhinoviruses, influenza viruses, West Nile virus, herpes simplex virus, HIV-1, HIV-2, adenovirus, cornavirus, influenza virus, rubella virus, yellow fever virus and respiratory syncytial virus (RSV). In a preferred embodiment, the viruses that may be inhibited by administration of the composition include at least Influenza A/Hong Kong/8/68 (H3N2).

Alternatively, the subject may be a member of the bird (Avian) species, which includes the common commercial poultry birds: turkeys, ducks, geese and chickens, less commonly the ostrich as well as other bird species that are commonly kept as house pets, for example canaries and parrots. The composition may be administered by directly spraying the composition into the nasal passage of the bird or the composition may be administered by creating a mist through which the birds walk. Thus, the composition may be given prophylactically to act in a virucidal or virustatic manner. Alternatively, the composition may be used to reduce the transmissivity of the virus.

The symptoms, caused by a viral infection, that may be treated, reduced, or at least partially prevented by this method of the present invention, may include one or more of headache, joint pain, fever, cough, sneezing, muscle ache, running nose, dry mouth, dizziness, and other symptoms related to viral infection. In birds, these symptoms include a lack of energy, decreased egg production, soft shelled eggs, a swelling of the head, eyelids, etc., nasal discharge, coughing or diarrhea.

The effective amount of the composition will vary depending on such factors as the subject being treated, the particular mode of administration, the activity of the particular active ingredients employed, the age, bodyweight, general health, sex and diet of the subject, time of administration, rate of excretion, the particular combination of ingredients employed, the total content of the main ingredient of the composition, and the severity of the illness or symptom. It is within the skill of the person of ordinary skill in the art to account for these factors.

When the composition is administered as a spray, the amounts each of the active ingredients may be reduced as the spray composition delivers the active ingredients more directly to the location where they are needed, as compared to a lozenge or capsule for example.

The following examples are intended to illustrate, but not to limit, the scope of the invention. Indeed, those of ordinary skill in the art can readily envision and produce further embodiments, based on the teachings herein, without undue experimentation. The scope of invention is to be determined by the claims appended hereto.

### EXAMPLE 1

### A Composition of the Present Invention

Pomegranate juice (PJ) concentrate (Wonderful variety, POM Wonderful, Los Angeles) was used in studies to confirm the benefits of the treatment set forth herein. Pomegranates were hand-picked, washed, chilled to 4° C., and stored in tanks. The fruit was then crushed, squeezed, and treated enzymatically with pectinase to yield the juice and byproducts, which included the inner and outer peels and the seeds. Pectinase hydrolyzes-1,4-galacturonide bonds in pectin, improving extraction and filtration, and prevents formation of pectin gels. Flavonoids constitute 40% (anthocyanins, catechins, and phenols) of total polyphenols in PJ. More complex polyphenols are also present in the juice. The PJ was filtered, pasteurized, concentrated, and stored at −20° C. until use.

### EXAMPLE 2

### Treatment of Sore Throat

Each of seven human subjects, suffering from sore throats, ingested one cup of PJ prepared according to Example 1 every four hours.

The human subjects that were treated reported complete relief from the symptoms of their sore throats after ingesting from 1 to 6 cups of PJ. It was also found that each cup of PJ can provide relief from a sore throat for up to 6 hours.

Transmission of cold/flu virus is from person to person usually by personal contact by shaking hands and wipe nose or rub eyes or getting sneeze on and the like.

Mechanism of infection: Upon entry through the nose and the eyes, the virus enters the cells lining the nasopharynx (the area of the upper throat that lies behind the nose) and multiples rapidly. Once viruses reach a high concentration cold/flu symptoms (related to inflammation) rapidly appear.

The nasopharynx is area typically for build high concentration of viruses needed to cause cold or flu symptoms. This build-up of viral concentration is because they are either: (1) trapped by the mucus membranes, which is a natural defense mechanism and/or (2) the viruses concentrate on the outer part of the membranes, where they are less exposed to normal body temperature (since they cannot survive elevated temperatures such as body temperature). The cold/flu viruses are vulnerable on the top of nasopharynx membranes on the upper throat.

Human subjects infected with cold/flu virus not showing symptoms are selected in this study. It is desirable keep the virus concentrations low in the upper throat/nasopharynx during the first few days of infection. This prevents the body's defense mechanism, e.g. sneezing, runny nose, fever, malaise and cough, from turning on as the symptoms may confound the study.

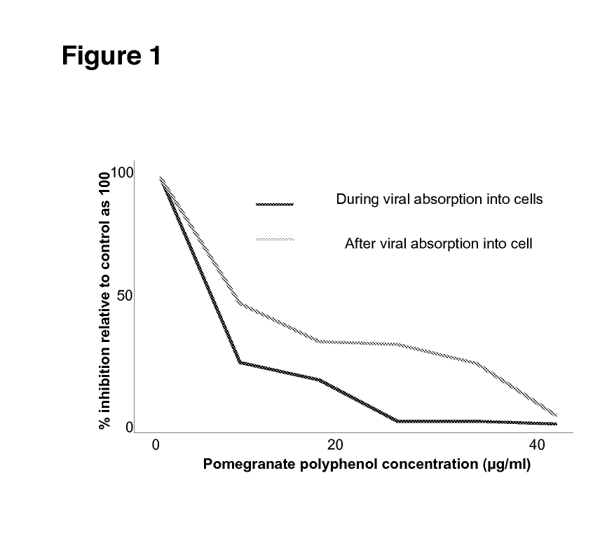

The oral consumption of PJ can strongly inhibit the viruses on the nasopharynx since it coats the lining of the throat/nasopharynx with PJ. The varying amount of PJ used to inhibit viral replication on both surface and inner layers of the cells are shown in FIG. 1.

It should be noted that the concentration of polyphenols in PJ is over 100 times more concentrated than in the diluted PJ as indicated in the FIG. 1. This high concentrated source of polyphenols in PJ can immediately inhibit viral replication, protein transport systems, and residual polyphenols are expected to remain for some period in on the cell surface or mucosal layer surrounding the throat/nasopharynx cells. So PJ is used to treat the nasopharynx which disrupt viral replication and control cold/flu symptom, typically when caught in time before viral levels get too high.

Conclusion: Daily or multi-times per day consumption of PJ may help keep viral concentrations at levels below threshold for symptoms. This study indicates prevention during the first 1-2 day of exposure.

What is claimed is:

1. A method of treating a subject with influenza comprising:
administering pomegranate extract in a dose sufficient to reduce or minimize symptoms in a subject having influenza.

2. A method for reducing one or more symptoms of an illness comprising:
administering to a subject that has been, or is likely to be, exposed to the illness, an amount of a composition comprising: pomegranate extract;
said amount being effective, when administered to reduce one or more of said symptoms of said illness or a severity of symptoms of said illness.

3. The method of claim 2, wherein said illness is caused by an influenza virus.
